(19) United States
(12) Patent Application Publication
(10) Pub. No.: US 2021/0338891 A1
(43) Pub. Date: Nov. 4, 2021

(54) DECELLULARIZATION OF TISSUES USING SUPERCRITICAL CARBON DIOXIDE

(71) Applicant: UNIVERSITY OF SOUTH CAROLINA, Columbia, SC (US)

(72) Inventors: DOMINIC M. CASALI, ELGIN, SC (US); MICHAEL A. MATTHEWS, COLUMBIA, SC (US)

(21) Appl. No.: 17/370,101

(22) Filed: Jul. 8, 2021

Related U.S. Application Data
(62) Division of application No. 15/810,904, filed on Nov. 13, 2017.
(60) Provisional application No. 62/471,028, filed on Mar. 14, 2017.

(51) Int. Cl. A61L 27/36

(52) U.S. Cl. CPC A61L 27/3687; A61L 27/3691; A61L 2430/40; A61L 27/3633; A61L 27/3625; A61L 27/3604

(57) ABSTRACT

A system and method for decellularizing tissue is provided. The system includes a pretreatment chamber including a pretreatment solution (e.g., a surfactant), a decellularization solution comprising carbon dioxide and one or more polar solvents, as well as an environmental chamber comprising a treatment chamber. The environmental chamber is maintained at a temperature greater than 31.1° C. and the carbon dioxide is maintained at a pressure greater than 7.38 megapascals to form supercritical CO₂. Tissue treated with the decellularization system and method can contain less than 0.05 micrograms of DNA per milligram of dry tissue after the tissue is exposed to the decellularization solution for a time period ranging from about 1 minute to about 2 hours with minimal ECM fiber disruption. A two-part decellularization solution comprising a surfactant as well as supercritical CO₂ and one or more polar solvents is also provided.

### RELATED APPLICATIONS

The present application is a divisional application of U.S. patent application Ser. No. 15/810,904, filed on Nov. 13, 2017, which claims priority to U.S. Provisional Application Ser. No. 62/471,028, filed on Mar. 14, 2017, the contents of both of which are incorporated herein in their entirety by reference thereto.

### BACKGROUND

Over 8,000 Americans die annually while awaiting an organ transplant, and currently, over 120,000 Americans are on the national transplant waiting list. Furthermore, the average transplant wait time is several years. One way to address this problem is the implantation of artificial tissues and organs created by tissue engineering (TE). This could drastically reduce wait times and alleviate the current dearth of available organ donors. However, tissues and organs are extraordinarily nuanced and complicated structures, presenting numerous requirements for creating effective biomimetic materials.

Whether derived from synthetic or natural materials, TE scaffolds must be sterile, porous, mechanically strong, and biocompatible and must also be of appropriate stiffness and surface chemistry for their specific application. Additionally, the scaffold fabrication process may introduce several structural and biochemical deficiencies, including loss of mechanical strength, loss of surface activity, denaturation of extracellular matrix (ECM) proteins, scaffold dehydration, and residual cytotoxicity of some solvents, detergents, and/or crosslinking agents. All of these challenges require continual development of innovative scaffold fabrication methods.

Additionally, TE scaffolds must direct cell proliferation and differentiation during tissue growth. This is a particular strength of naturally derived biomaterials, which have recently been shown to promote constructive remodeling during tissue growth. Scaffolds prepared from decellularized tissues are uniquely able to receive and transmit signals to cells, an interaction known as dynamic reciprocity. Decellularized ECM has also been shown to elicit an anti-inflammatory immune response, which may be related to a reduced risk of rejection.

Decellularization can be accomplished using a variety of techniques, including physical, chemical, and enzymatic treatment methods. Treatment with aqueous detergents, such as sodium dodecyl sulfate (SDS), is most common. Detergents lyse cell and nuclear membranes, but also denature proteins, which can lead to thorough cell removal but can also disrupt glycosaminoglycans (GAGs), growth factors, and ECM ultrastructure. Because of these hazards, it has become common to use detergents at very low concentrations over multiple days or even weeks, avoiding the ECM drawbacks while eventually removing all cells. Though this approach is effective, improved methods would be useful to decellularize tissues as effectively but with shorter treatment times and without using harsh chemicals or solvents for long periods.

One method contemplates decellularization with supercritical carbon dioxide (CO₂). CO₂ is non-toxic, non-flammable, and relatively inert; it has desirable solvent properties and a mild critical temperature (31.1° C.), making it viable at physiological temperatures. CO₂ has been used extensively in TE applications such as polymer foaming, where CO₂ is used to fabricate TE scaffolds from synthetic biomaterials. Supercritical CO₂ has also been utilized in other biomedical applications, including extraction of biologically relevant molecules, as well as pasteurization and sterilization of synthetic biomaterials. However, little work exists for CO₂ and natural biomaterials.

An improved supercritical CO₂ decellularization treatment that would allow for reduced decellularization treatment times, such as on the order of hours instead of days, would be useful. The absence of detergents could also reduce damage to the ECM. While studies have shown that supercritical CO₂ decellularization can lead to adequate DNA and cellular removal, supercritical fluid (SCF) extraction of volatile substances during treatment, primarily water, can result in hardening of the tissue and subsequent scaffold embrittlement, potentially endangering the viability of the scaffold. This raises a significant obstacle to progress in the field.

Other decellularization protocols that utilize ethanol have reported similar tissue dehydration, so the reported extraction of water and volatiles during treatment with CO₂ and ethanol is not surprising. In fact, this phenomenon is very similar to critical point drying, which is commonly used in tissue engineering and other applications, such as electronics processing and scanning electron microscopy. However, in this application, it is desirable to avoid the drying phenomenon entirely. It is hypothesized that dehydration caused by CO₂ treatment can be significantly reduced or even eliminated by presaturating supercritical CO₂ with water and other biological volatiles prior to treatment. Establishing this thermodynamic equilibrium will prevent volatile components from being extracted.

As such, what is needed is an improved supercritical CO₂ decellularization method that also maintains the hydration state of the treated tissue. The objectives are as follows: (1) to develop a system and method that can presaturate supercritical CO₂ with water; (2) to treat two model scaffolds (a model hydrogel and porcine aorta) with dry and presaturated CO₂, and compare the level of dehydration observed; (3) to examine the extent of decellularization in porcine aorta using CO₂ with different additives, pretreatments, and thermodynamic conditions; and (4) to present a hybrid CO₂/detergent treatment that decellularizes the tissue more quickly and as effectively as a standard detergent treatment that can require up to about 10 days of PBS washing to remove residual SDS surfactant, where the presence of residual SDS can disrupt the ECM fibers in decellularized tissue. Achieving these objectives would enable further development of CO₂-based tissue engineering and decellularization processes.

### SUMMARY OF THE INVENTION

In one embodiment of the present invention, a system for decellularizing tissue is provided. The system includes a pretreatment chamber comprising a pretreatment solution, wherein the pretreatment solution comprises a surfactant, a decellularization solution comprising carbon dioxide and one or more polar solvents; and an environmental chamber comprising a treatment chamber, wherein the environmental chamber is maintained at a temperature greater than 31.1° C. and the carbon dioxide is maintained at a pressure greater than 7.38 megapascals to form supercritical carbon dioxide.

In another embodiment, the treatment chamber can receive the tissue.

In one embodiment, the environmental chamber can include a presaturation chamber, wherein the decellularization solution can be mixed in the presaturation chamber. Further, the decellularization solution can be deliverable from the presaturation chamber to the treatment chamber.

In still another embodiment, the system can include a pump, wherein the pump compresses the carbon dioxide.

In yet another embodiment, the decellularization solution can be delivered to the treatment chamber at a flow rate ranging from about 0.1 millimeters per minute to about 5 milliliters per minute.

In an additional embodiment, the system can facilitate removal of cells from the tissue so that tissue treated with decellularization solution contains less than 0.05 micrograms of DNA per milligram of dry tissue after the tissue is exposed to the decellularization solution.

In another particular embodiment, the surfactant wash can include sodium dodecyl sulfate.

In an additional embodiment, the one or more polar solvents can include ethanol, methanol, isopropanol, water, acetic acid, or a combination thereof.

In one more embodiment of the present invention, a method for decellularizing tissue is provided. The method includes pretreating the tissue with a surfactant and treating the tissue with a decellularization solution comprising carbon dioxide and one or more polar solvents at a temperature greater than 31.1° C., wherein the carbon dioxide is maintained at a pressure greater than 7.38 megapascals to form supercritical carbon dioxide.

In one embodiment, a syringe pump can compress the carbon dioxide before the carbon dioxide is delivered to the tissue.

In another embodiment, the carbon dioxide and the one or more polar solvents can be mixed for a time period ranging from about 1 minute to about 30 minutes prior to exposing the tissue to the decellularization solution. Further, the one or more polar solvents can include ethanol, methanol, isopropanol, water, acetic acid, or a combination thereof.

In an additional embodiment, the decellularization solution can be delivered to a treatment chamber containing the tissue. Moreover, the decellularization solution can be delivered to the treatment chamber at a flow rate ranging from about 0.1 millimeters per minute to about 5 milliliters per minute.

In one more embodiment, the decellularization solution can be mixed in a presaturation chamber, and the decellularization solution can be delivered from the presaturation chamber to a treatment chamber containing the tissue.

In still another embodiment, the tissue can be exposed to the decellularization solution for a time period ranging from about 1 minute to about 2 hours.

In yet another embodiment, the surfactant can include sodium dodecyl sulfate.

In an additional embodiment, the method can facilitate removal of cells from the tissue so that tissue treated with decellularization solution contains less than 0.05 micrograms of DNA per milligram of dry tissue after the tissue is exposed to the decellularization solution.

In one more embodiment of the present invention, a two-part decellularization solution for removing cells from tissue is provided. A first part of the two-part decellularization solution includes a surfactant, and a second part of the two-part decellularization solution includes supercritical carbon dioxide and one or more polar solvents.

In one embodiment, the surfactant can include sodium dodecyl sulfate and the one or more polar solvents can include ethanol, methanol, isopropanol, water, acetic acid, or a combination thereof.

In another embodiment, the tissue treated with two-part decellularization solution can contain less than 0.05 micrograms of DNA per milligram of dry tissue after the tissue is exposed to the two-part decellularization solution for a time period ranging from about 1 minute to about 2 hours. Moreover, tissue treated with the two-part decellularization solution contains less than 0.0045 volume % of residual surfactant after the issue is exposed to the second part of the two-part decellularization solution.

Other features and aspects of the present invention are set forth in greater detail below.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying Figures.

FIG. 1 shows a schematic of a presaturation system according to one particular embodiment;

FIG. 2 is a graph showing the observed mole fraction/equilibrium solubility versus CO₂ mass flow rate in grams/minute, showing that complete presaturation of the CO₂ is achieved at low flow rates;

FIG. 3 is a graph showing the % mass retained of a hydrogel at 37° C., a hydrogel at 50° C., and a porcine aorta at 37° C. when treated with dry carbon dioxide and presaturated carbon dioxide;

FIG. 4 is a graph showing the decrease in dimensionless mass over time as a result of vacuum drying a porcine aorta at 37° C. and 38.1 centimeters of mercury (cm Hg) compared to dry CO₂ and presaturated CO₂;

FIG. 5 shows hemotoxylin and eosin (H&E) stained sections of untreated (a, d), SDS-treated (b, e), and dry CO₂-treated (c, f) porcine aorta at low (top row) and high (bottom row) magnification, where the scale bars represent 50 micrometers;

FIG. 6 shows hemotoxylin and eosin (H&E) stained sections of untreated (a,d), water/CO₂-treated (b, e), and water/Ls-54/CO₂-treated (c, f) porcine aorta at low (top row) and high (bottom row) magnification, where the scale bars represent 50 micrometers;

FIG. 7 shows hemotoxylin and eosin (H&E) stained sections of untreated (a, d), ethanol/CO₂-treated (b, e), and ethanol/water/CO₂-treated (c, f) porcine aorta at low (top row) and high (bottom row) magnification, where the scale bars represent 50 micrometers;

FIG. 8 is a graph showing the DNA concentration in micrograms/milligram of porcine aorta decellularized via various methods, where values below the horizontal line indicate complete decellularization;

FIG. 9 shows hemotoxylin and eosin (H&E) stained sections of untreated (a, d), SDS-treated (b, e), and SDS/CO₂-treated (c, f) porcine aorta at low (top row) and high (bottom row) magnification, where the scale bars represent 50 micrometers;

FIG. 10 is a graph showing the DNA concentration in micrograms/milligram of porcine aorta decellularized via various methods, where values below the horizontal line indicate complete decellularization; and

FIG. 11 is a graph showing the results of a residual sodium dodecyl sulfate (SDS) quantitation assay, where SDS was quantified before and after washing with either PBS or supercritical CO₂, where 1 hour of supercritical CO₂ treatment compares similarly to 24 hours of PBS washing.

Repeat use of reference characters in the present specification and drawings is intended to represent the same or analogous features or elements of the present invention.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

The present invention is directed to a system and method for decellularizing tissue (e.g., porcine aorta) for use in tissue engineering applications using supercritical CO₂ and one or more polar solvents (e.g., ethanol, methanol, isopropanol, water, acetic acid, or a combination thereof). The present invention is also directed to a decellularization solution for removing cellular matter from the tissue. The system, method, and decellularization solution of the present invention can allow for decellularization of a native scaffold while maintaining ECM fiber integrity, which is a problem seen during conventional detergent and saline washing methods. Further, the system and method can include a pretreatment solution or wash that includes a surfactant such as sodium dodecyl sulfate. In some embodiments, the pretreatment solution can be referred to as a first part of the decellularization solution, and the supercritical CO₂ and the one or more polar solvents (e.g., ethanol, methanol, isopropanol, water, acetic acid, or a combination thereof) can be referred to collectively as a second part of the decellularization solution, where the tissue can be exposed to the first part of the decellularization solution (e.g., the surfactant) in a separate chamber from the second part of the decellularization solution. The system and method facilitate removal of cells from the tissue so that tissue treated with decellularization solution contains less than 0.05 micrograms of DNA per milligram of dry tissue after the tissue is exposed to the decellularization solution.

Additionally, exposure to supercritical CO₂ at the conditions contemplated by the present invention can result in high-level disinfection of the tissue being decellularized and can remove microbial contaminants from the tissue. Supercritical CO₂ is formed when pure CO₂ is heated and pressurized above the critical conditions of 31.1° C. and 7.38 megapascals (MPa). In particular, the system contemplated by the present invention is a dynamic carbon dioxide flow system that is used to extract cellular matter from tissue such as porcine aorta. Furthermore, exposure to supercritical CO₂ at the conditions used is known to cause high-level disinfection, removing microbial contaminants from the tissue.

The system and method of the present invention contemplate the use of high-pressure vessels, which can be made of stainless steel or any other suitable material, an environmental chamber, a back-pressure regulator, and a pump (e.g., a syringe pump or any other suitable pump). In one particular embodiment, the environmental chamber, back-pressure regulator, and syringe pump maintain constant conditions within the system. For instance, the environmental chamber can be maintained at a temperature greater than about 31.1° C. as required to form supercritical CO₂. For instance, the temperature can range from about 32° C. to about 42° C., such as from about 34° C. to about 40° C., such as from about 36° C. to about 38° C. Further, the pressure in the system can be maintained at a level above about 73.8 bar (7.38 megapascals) as required to form supercritical CO₂. For example, the pressure in the system can range from about 95 bar (9.5 megapascals) to about 350 bar (35 megapascals), such as from about 97.5 bar (9.75 megapascals) to about 300 bar (30 megapascals), such as from about 100 bar (10 megapascals) to about 280 bar (28 megapascals). In addition, the CO₂ flow rate can range from about 0.1 milliliters per minute to about 5 milliliters per minute, such as from about 0.2 milliliters per minute to about 3 milliliters per minute, such as from about 0.5 milliliters per minute to about 2.5 milliliters per minute. In other words, for a treatment chamber having a volume of 10 milliliters, for example, the residence time of the CO₂ decellularization solution can range from about 2 minutes to about 100 minutes, such as from about 3 minutes to about 50 minutes, such as from about 4 minutes to about 20 minutes.

In one particular embodiment, the environmental chamber can be maintained at a temperature of about 37° C., the pressure can be maintained between 100 bar (10 megapascals) and 300 bar (30 megapascals), and the CO₂ flow rate can be maintained at about 1 milliliter per minute. In an additional embodiment, the environmental chamber can include a first pressure vessel (e.g., a presaturation chamber) and a second pressure vessel (e.g., a treatment chamber), where CO₂ is continually mixed with the polar solvent (e.g., ethanol and water) using a magnetic stirrer to form a CO₂/polar solvent decellularization solution. The resulting decellularization solution is then introduced into the second pressure vessel, where it removes cellular material that is present in the tissue. However, it is also to be understood that the present invention contemplates an environmental chamber that includes a single pressure vessel (e.g., a treatment chamber), where the CO₂/polar solvent decellularization solution is delivered to the treatment chamber with, for example, two pumps (e.g., syringe pumps). In such an embodiment, one pump delivers the liquid CO₂ to the treatment chamber and the other pump delivers the polar solvent(s) to the treatment chamber at predetermined ratios to deliver the appropriate concentration of each component of the decellularization solution to the treatment chamber. In another embodiment, it is contemplated that an injection loop can be used to deliver the decellularization solution directly to the treatment chamber.

The system and method also contemplate the use of a pretreatment solution containing a surfactant to pretreat the tissue in a pretreatment chamber, where the pretreated tissue is then subjected to treatment with the aforementioned decellularization solution and conditions in a separate treatment chamber. The pretreatment solution can include sodium dodecyl sulfate.

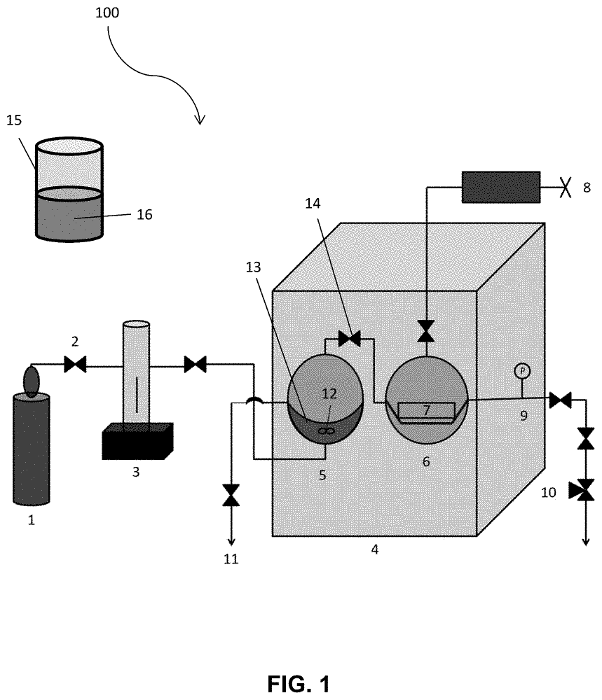

Specifically, and referring to FIG. 1, in one particular embodiment, the decellularization system 100 of the present invention can include a supply of liquid carbon dioxide 1, a high-pressure valve 2, a pump 3 (e.g., a syringe pump), an environmental chamber 4, a presaturation chamber 5 containing a stir bar 12 to mix the CO₂ and polar solvent (e.g., ethanol and water) to form a decellularization solution 13, a treatment chamber 6 containing the tissue to be decellularized (e.g., a porcine aorta) 7, a CO₂ hand pump 8, a pressure gauge 9, a back pressure regulator 10, a treatment chamber valve 14, and an emergency vent 11. The system can also include a pretreatment chamber 15 that can include a pretreatment solution 16. In one embodiment, the pretreatment solution 16 can be a surfactant such as sodium dodecyl sulfate (SDS).

Generally, to decellularize the tissue 7, such as a porcine aorta, the tissue 7 is loaded into the treatment chamber 6 of the decellularization system 100. The treatment chamber 6 is located in an environmental chamber 4. Then, liquid carbon dioxide 1 can be compressed in a chilled syringe pump 3 or any other suitable pump and slowly bubbled into a first high-pressure vessel, which can be referred to as the presaturation chamber 5, which is also located in the environmental chamber 4. In the presaturation chamber 5, additives (e.g., one or more polar solvents including water and ethanol) can be mixed with the carbon dioxide 1 using a stir bar 12 until the one or more polar solvents is fully dissolved in the carbon dioxide 1 to form the decellularization solution 13. The carbon dioxide 1 and additive(s) (e.g., the one or more polar solvents) can be mixed for a time period ranging from about 1 minute to about 30 minutes, such as from about 5 minutes to about 25 minutes, such as from about 10 minutes to about 20 minutes. In one particular embodiment, the carbon dioxide and additive(s) (e.g., the one or more polar solvents) can be mixed for a time period of about 10 minutes to about 15 minutes. Next, the valve 14 to the treatment chamber 6, which contains the tissue 7 to be decellularized, can be opened, and the CO₂ flow through the treatment chamber 6 at a rate ranging from about 0.1 milliliters per minute to about 5 milliliters per minute, such as from about 0.2 milliliters per minute to about 3 milliliters per minute, such as from about 0.5 milliliters per minute to about 2.5 milliliters per minute. In one particular embodiment, the CO₂ flow rate through the treatment chamber 6 can be about 1 milliliter per minute. In addition, the tissue 7 can be treated for a time frame ranging from about 1 minute to about 2 hours, such as from about 2 minutes to about 90 minutes, such as from about 4 minutes to about 1 hour. In one particular embodiment, the tissue 7 can be treated for about 1 hour.

It is also to be understood that the surfactant pretreatment step mentioned above can be carried out by placing the tissue 7 in the pretreatment chamber 15 prior to exposing the tissue 7 to the carbon dioxide and one or more polar solvents of the decellularization solution 13 being introduced into the treatment chamber 6. For instance, the tissue 7 can be pretreated with the pretreatment solution 16 (e.g., surfactant) for a time frame ranging from about 8 hours to about 72 hours, such as from about 12 hours to about 60 hours, such as from about 24 hours to about 58 hours, after which the tissue 7 can then be placed in the treatment chamber 6. In one particular embodiment, the tissue 7 can be pretreated with the surfactant for about 48 hours.

Moreover, the temperature can be maintained at a temperature greater than 31.1° C. (e.g., at 37° C. or 50° C.) by the environmental chamber 4 and the pressure of the CO₂ in the vessels can be maintained at a level greater than about 7.38 megapascals (e.g., at about 10.3 megapascals or at about 27.6 megapascals) using a 6000 psi back-pressure regulator 10 in order to maintain the carbon dioxide 1 in a supercritical state. A manual hand pump 8 can be used to depressurize the system 100 at a rate ranging from about 0.1 megapascals per minute to about 0.6 megapascals per minute, such as from about 0.2 megapascals per minute to about 0.5 megapascals per minute, such as from about 0.3 megapascals per minute to about 0.4 megapascals per minute. For instance, the system 100 can be depressurized at a rate of about 0.345 megapascals per minute (50 psi/minute). It is to be understood that various valves (e.g., high pressure valve 2, valve 14) and fittings (e.g., back pressure regulator 10) rated for pressures up to 68.9 MPa can be used throughout the system 100.

After treatment with the decellularization solution of the present invention using the system and method described herein, tissue treated with decellularization solution can contain less than 0.05 micrograms of DNA per milligram of dry tissue after the tissue is exposed to the decellularization solution. For instance, tissue treated with decellularization solution can contain from about 0.01 micrograms of DNA per milligram of dry tissue to about 0.05 micrograms of DNA per milligram of dry tissue, such as from about 0.015 micrograms of DNA per milligram of dry tissue to about 0.045 micrograms of DNA per milligram of dry tissue, such as from about 0.02 micrograms of DNA per milligram of dry tissue to about 0.04 micrograms of DNA per milligram of dry tissue after the tissue is exposed to the decellularization solution.

Further, after treatment with the decellularization solution of the present invention using the system and method described herein, tissue treated with decellularization solution can contain less than 0.0045 volume % of surfactant (e.g., sodium dodecyl sulfate) when pretreated with a surfactant as described in the present invention. For instance, tissue treated with decellularization solution can contain from about 0.001 volume % to about 0.004 volume %, such as from about 0.00125 volume % to about 0.0038 volume %, such as from about 0.0015 volume % to about 0.0034 volume % of surfactant after the tissue is exposed to the decellularization solution.

The present invention may be better understood with reference to the following example.

EXAMPLE 1

Example 1 demonstrates the ability to decellularize tissue in an efficient and effective manner according to the system and method contemplated by the present invention.

### Materials and Methods

### Apparatus Development and Validation

To prevent water extraction from porcine tissue it is necessary to first achieve dynamic thermodynamic equilibrium (i.e., complete saturation) between CO₂ and water. The saturated CO₂ phase is subsequently suitable for treating a TE matrix. The first experimental objective was to ensure that the CO₂ was being fully saturated during the mixing process. Achieving this goal was critical before attempting to decellularize any tissue samples.

A schematic of the presaturation apparatus is shown in FIG. 1. Liquid carbon dioxide 1 (bone-dry grade with siphon tube, 99.8% purity, Praxair Inc., Danbury, Conn.) was compressed in a chilled syringe pump 3 (500 HP Series, ISCO Inc., Lincoln, Neb.) and slowly bubbled into the presaturation chamber 5 (Waters Corp., Milford, Mass.), where 10 milliliters of water was previously added. In this chamber, CO₂ and the additive (e.g., ethanol and water) were stirred vigorously using a stir bar 12 until reaching thermodynamic equilibrium (about 15 min) to form a decellularization solution 13. At this point, the valve 14 to the treatment chamber 6 was opened and flow was introduced into the 10-milliliter treatment chamber 6, which contained the tissue 7 to be deceullularized. Constant flow was maintained by the syringe pump 3 at 1 mL/min for the desired treatment time (usually about 1 hour).

The temperature was maintained at the desired temperature (e.g., 37° C. or 50° C.) by the environmental chamber 4 (LU-113 model, ESPEC Corp., Osaka, Japan), and the pressure of the supercritical CO₂ in the vessels was maintained at 13.79 megapascals (2000 psi) using a 6000 psi back-pressure regulator 10 (TESCOM, Elk River, Minn.). A manual hand pump 8 (HiP Pressure Generator 62-6-10, High Pressure Co.) was used to depressurize the system at a rate of 0.345 megapascals/minute (50 psi/minute). Valves and fittings rated for pressures up to 68.9 megapascals (High Pressure Co., Erie, Pa.) were used throughout the system (e.g., high pressure valve 2, valve 14, back pressure regulator 10, etc.) rated for pressures up to 68.9 MPa can be used throughout the system 100.

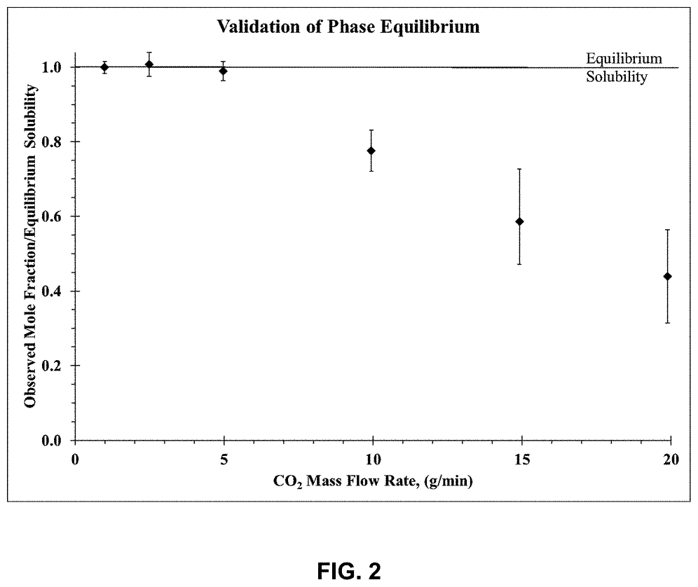

A preliminary test using a cold trap to collect dissolved water in the effluent showed complete thermal equilibrium at flow rates of 5 milliliters of CO₂ per minute and below, as measured at the pump inlet. This data is presented in FIG. 2. At flow rates of 5 milliliters per minute and below, the observed water mole fractions approach the equilibrium limit. As the flow rate increases, the observed mole fraction decreases, indicating failure to equilibrate. As such, CO₂ flow rates of 1 milliliter per minute were used for the remainder of this work.

### Biomaterial Selection and Preparation

To further validate the overall presaturation concept, a synthetic biomaterial (a hydrogel) and a natural tissue, porcine aorta, were utilized. The hydrogel was poly(acrylic acid-co-acrylamide) potassium salt (Sigma-Aldrich, St. Louis, Mo.), a hydrogel used previously to establish the ability of CO₂ to achieve sterilization. Hydrogel powder was hydrated in excess water at 4° C. for 24 hours. Excess water was removed from each hydrogel specimen by drying for 30 minutes under a light vacuum, using filter paper and a Buchner funnel. Each hydrogel was blotted onto a nylon filter and sealed inside the treatment chamber prior to the start of each trial. The weight of each gel was approximately 0.2 grams.

Porcine aorta was obtained from a local slaughterhouse and the surrounding fatty tissue was removed. The aortic tissue was cut into thin rectangles (approximately 3 centimeters by 2 centimeters) and stored in phosphate-buffered saline (PBS) at 4° C. for up to 48 hours prior to use. Each tissue specimen was dried for 15 minutes under a light vacuum using filter paper and a Buchner funnel to remove free saline prior to weighing and treatment. Drying in a vacuum oven (37° C., 38.1 centimeters of mercury (cm Hg) vacuum) was used as a negative control; changes in mass were recorded after 1, 2, 3, 6, and 24 hours. The treatment ratio and other conditions used (including temperature, pressure, and depressurization rate) were chosen to be analogous to the conditions used by K. Sawada, et al. in “Cell removal with supercritical carbon dioxide for acellular artificial tissue,” Journal of Chemical Technology and Biotechnology, 83 (2008) 943-949, to allow for comparison.

### Dehydration of Model Matrix Materials

All treatments were performed using the apparatus shown in FIG. 1. In these tests, a hydrated (or native) biomaterial was weighed, then contacted with either dry CO₂ or pre-saturated CO₂. After treatment, the biomaterials were weighed again, and changes in mass and overall appearance were recorded. Two treatments were conducted on each biomaterial: one using dry CO₂ (no water in presaturation chamber) and the other using CO₂ presaturated with water. All treatments were carried out at 13.79 MPa (2000 psi). The temperature was held constant at either 37° C. (ρ_{CO2}=0.769 g/mL) or 50° C. (ρ_{CO2}=0.665 g/mL, for hydrogel only). Four duplicate treatments were made at each temperature. All biomaterials, regardless of initial mass, were treated using a treatment ratio of 30 minutes of CO₂ flow per 0.1 gram of gel or tissue, or a treatment time of approximately 1 hour for all specimens.

Standard Decellularization with SDS

For decellularization treatments, porcine aorta was obtained from a local abattoir, rinsed in phosphate buffered saline (PBS) and cut into ring-shaped sections measuring about 1 centimeter in width. At this point, tissues were stored at −20° C. until treatment.

The standard SDS treatment is described as follows. Briefly, tissue was first immersed and agitated for 1 hour in a solution containing 0.2% (w/v) EDTA and 10 mM pH 8 Tris buffer to increase cell membrane permeability. It was then decellularized for 48 hours under agitation in 0.1% (v/v) SDS, 10 mM Tris buffer, 0.2 mg/mL DNase I, and 0.02 mg/mL RNase. Matrices were washed with PBS several times over the course of 24 hours to remove cell debris and residual detergent.

Decellularization with Supercritical CO₂

Tissue was loaded into the treatment chamber of the supercritical CO₂ apparatus as described in FIG. 1. Liquid carbon dioxide (bone-dry grade with siphon tube, 99.8% purity, Praxair Inc., Danbury, Conn.) was compressed in a chilled syringe pump (500 HP Series, ISCO Inc., Lincoln, Neb.) and slowly bubbled into the presaturation chamber (Waters Corp., Milford, Mass. to maximize mass transfer. In this chamber, the additive and CO₂ were stirred vigorously until reaching thermodynamic equilibrium (usually 10-15 minutes). Four different solutions were used in the presaturation chamber in order to determine whether aqueous additives enhanced decellularization: water, water plus Dehypon® Ls-54 Surfactant (BASF America, Florham Park, N.J.), pure ethanol, and water plus ethanol.

Once equilibrium was reached, the valve to the treatment chamber, which contained the porcine aorta, was opened and CO₂ flow was programmed to 1 milliliter per minute at the pump inlet. During treatment, the environmental chamber (LU-113 model, ESPEC Corp., Osaka, Japan) was used to maintain the temperature at either 50° C. or 37° C., and a back-pressure regulator (TESCOM, Elk River, Minn.) was used to keep the CO₂ pressure in the vessels constant at either 10.3 or 27.6 MPa (1500 or 4000 psi). A manual hand pump (Pressure Generator 62-6-10, High Pressure Co.) was used to depressurize the system at a rate of 0.345 MPa/min (50 psi/min).

### Hematoxylin and Eosin (H&E) Staining

After CO₂ treatment, tissues were fixed in 10% neutral buffered formalin for at least 24 hours and embedded in paraffin. Tissues were then cut into 5 μm sections using a microtome and deparaffinized by immersion in xylene (3 times), 100% ethanol, 95% ethanol, 80% ethanol, and finally water. The tissues were stained with hematoxylin for 7 minutes, washed with water and ammonia, and then stained with eosin for 2 minutes before being dehydrated by immersion in 80% ethanol, 95% ethanol, 100% ethanol, and finally xylene (3 times). A coverslip was mounted on slides, which were then viewed using a light microscope (Nikon E600, Tokyo, Japan) after waiting at least 24 hours for the slides to dry.

### DNA Quantification

DNA quantification was performed with the DNAzol® reagent (Invitrogen, Carlsbad, Calif.) according to the prescribed protocol with minor changes. 25 milligrams of treated or untreated tissue was flash-frozen in liquid nitrogen and ground with a mortar and pestle. It was then placed in a 2-milliliter tissue homogenizer (VWR International, Radnor, Pa.) with 0.5 milliliters of DNAzol® reagent and ground for about 10 strokes or until fully dissolved. The solution was centrifuged at 10,000×g for 10 minutes and the supernatant was recovered. 0.25 milliliters of 100% ethanol were added to precipitate the DNA, which was recovered and washed twice with 70% ethanol for 1 minute per wash. DNA was next air-dried for 5 seconds and dissolved in a sodium hydroxide solution (pH 9). Absorbance at 260 nm was recorded using a spectrophotometer (DU 730 model, Beckman-Coulter, Brea, Calif.) and the DNA concentration was calculated based on the absorbance measurement and initial mass of the tissue.

### Hybrid SDS/CO₂ Treatment

After analyzing the results of the above treatments, development of a hybrid treatment was desired. The hybrid treatment included a shortened version of the standard SDS detergent treatment described above followed by CO₂ treatment described above, using water and ethanol together as the additives. Separate staining and other analyses were performed on the tissues treated with the hybrid method.

### Residual SDS Quantitation

Next, residual SDS from the standard and hybrid treatments was quantified using an SDS Detection and Estimation Kit (G Biosciences, St. Louis, Mo.). The assay involved mixing 1 mL of methylene blue dye with 0.5 mL extraction buffer and 5 μL of aqueous solution containing SDS, then vortexing for 30 seconds. 1 mL of chloroform was then added, then the mixture was vortexed again for 30 seconds. Methylene blue is extracted into the organic phase if SDS is present. After waiting 5 minutes, the bottom chloroform phase was sampled and optical density was measured at 600 nm. SDS concentration was calculated by comparison to a standard curve.

### Statistical Analysis

Numerical data is presented as mean values plus or minus one standard deviation. A student's t-test was used to analyze confidence in statistical differences between groups. 95% confidence (p<0.05) was considered to be statistically significant, while 99% confidence (p<0.01) was considered highly significant.

### Results and Discussion

### Dehydration of Matrix Materials

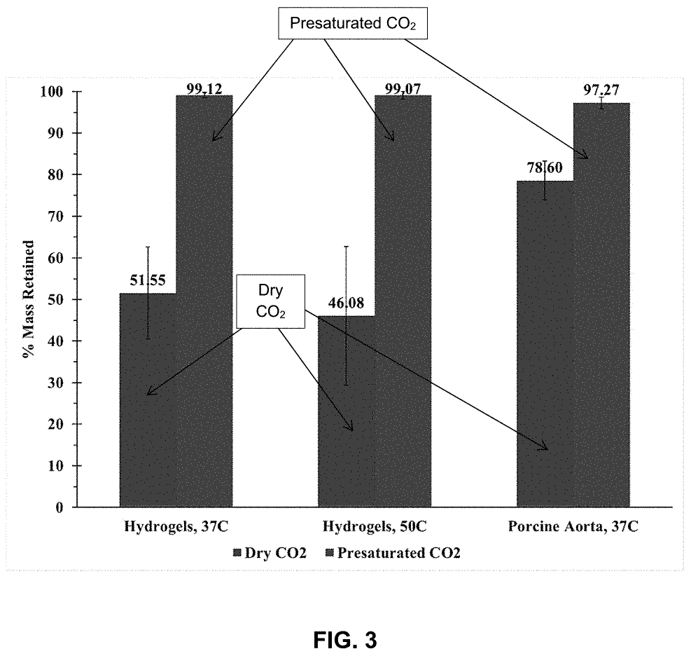

Hydrogels were treated with dry (control) and presaturated supercritical CO₂ at 37° C. and 50° C. and at 13.8 megapascals (2000 psi); porcine aorta was treated at 37° C. only. Data from these experiments are summarized in FIG. 3. For hydrogels at both temperatures, the average water retention was about 50% for dry CO₂, and just over 99% with presaturated CO₂. The differences in mean water retention at both temperatures were extremely significant.

Results when treating the hydrogel with supercritical CO₂ confirm the initial hypothesis. The control runs with dry CO₂ showed the expected dehydration of the hydrogels over time, caused by extraction of water by the dry CO₂, resulting in about a 50% average mass loss. The mass retention is slightly lower at 50° C., likely because both water vapor pressure and solubility in CO₂ increase with temperature, which would cause more water to be extracted at 50° C. than at 37° C. On the other hand, presaturated CO₂ extracts little to no water from the hydrogels, as over 99% of the biomaterial mass is maintained at both temperatures. This is a very strong indication that the presaturating the CO₂ with water prevents extraction, as predicted. One noticeable feature of FIG. 3 is that, in comparison to the other CO₂ treatment data presented, the error bars for dry CO₂ treatment of the hydrogels are quite large. This is likely related to structural changes in the hydrogel as it dries. Regardless of the specific amount of water lost during dry CO₂ treatment, FIG. 3 clearly indicates that using presaturated CO₂ prevents any considerable amount of water from being extracted from the hydrogels.

Results for the control (dry CO₂) and presaturated supercritical CO₂ treatments of porcine aorta are also shown in FIG. 3. The average mass retentions are 78.6%±4.6% with dry CO₂ and 97.3%±1.4% with presaturated CO₂; this difference is highly significant. It is evident from these results that using presaturated CO₂ considerably reduces the amount of mass lost during treatment, as expected based on theory and the analogous hydrogel results.

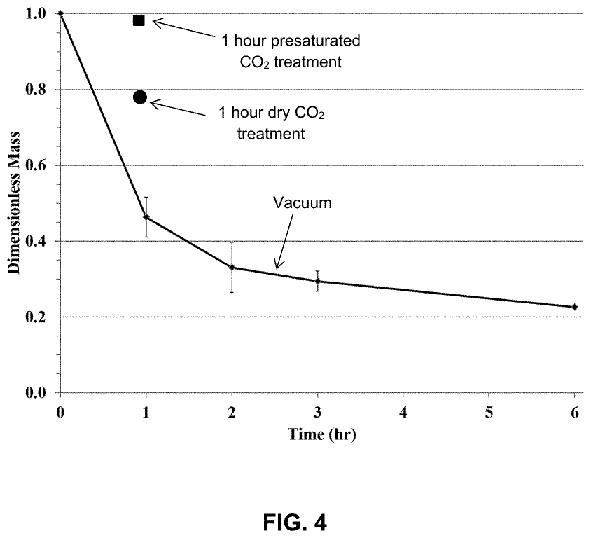

In addition, vacuum drying was used to produce a complete drying curve for porcine aorta tissue (n=6), as shown in FIG. 4, where dimensionless mass on the y-axis is the ratio of the mass measured at each time point to the original mass of the tissue. Substantial water loss occurred during the first six hours, but after six hours, the loss of mass was insignificant. Vacuum drying of the native tissue removed over half of the initial mass in the first hour, with a continually more gradual decline in mass over the next five hours until reaching slightly above 20% of the initial mass. This is consistent with the known water content of porcine aorta, which is about 75%. However, both the presaturated CO₂ and vacuum drying data suggest that a small amount (about 2-3% total mass) of substances other than water are extracted from the tissue. While the aorta is primarily composed of water and CO₂-insoluble proteins like elastin and collagen, porcine aorta also contains small amounts of lipids, such as cholesterol; these may comprise the remaining extracted substances in the tissue, as CO₂ has been proven efficacious for the extraction of fatty acids and other lipids. In the future, these substances could be identified by gas chromatography or similar methods. If maintaining their presence is important, their removal could be prevented in a similar manner to how water extraction is prevented in this study.

FIG. 4 also shows where dry and presaturated CO₂ treatment each lie on the tissue drying curve, as indicated by the circle and square, respectively. Comparison to CO₂ treatment shows that even dry CO₂ treatment causes much less drying than a vacuum treatment over the same time interval. This finding was also confirmed visually, as vacuum drying caused tissues to become significantly darker in color and far more brittle than those treated with CO₂. CO₂ drying is less severe than oven drying for two reasons. First, there is no mixing in the treatment chamber, and the flow rate is too high for equilibrium to be established between the water in the fluid phase and the tissue matrix. Second, there is also mass transfer resistance within the tissue, which slows transfer of water from the tissue to the flowing CO₂. However, though CO₂ drying is less drastic than vacuum drying, unintentional water extraction on this level could still significantly hamper the effectiveness of a TE scaffold, so minimizing even the smaller extent of drying caused by CO₂ is extremely important for TE applications.

Finally, it should be noted that this work is focused on preventing unintentional drying during CO₂ treatment, and the conclusion that any scaffold dried during a CO₂ process or otherwise is immediately invalid or not viable is not being made. In fact, work has recently been published by other groups where supercritical CO₂ is used to intentionally dry a TE scaffold. However, this has been done either with a scaffold material other than a decellularized tissue, and/or was done with the intention of long-term scaffold storage. After long-term storage, a scaffold would require rehydration before seeding and implantation. In addition to adding another processing step, the rehydration process has been shown to not fully restore the original water content of the matrix because of irreversible changes in ECM microstructure. Therefore, it is maintained that in producing a decellularized tissue for immediate use as a TE scaffold, it would be preferable to retain the original hydration state of the tissue after treatment.

Decellularization with Supercritical CO₂

With a method of preventing tissue dehydration now established, attention is now turned to decellularizing a tissue. The objective of decellularization is to maximize removal of cells and cellular debris while minimizing ECM alteration. A CO₂-based decellularization treatment would quicken the process compared to protocols that require weeks-long wash steps while using a benign solvent that leaves no residual material in the matrix.

Currently, there is no universally accepted standard for evaluating the extent of decellularization. This is not surprising because tissues vary greatly in stiffness, cell density, ECM composition, and numerous other characteristics, so decellularization processes must be tailored to the specific tissue of interest. However, a list of three criteria that can adequately describe a decellularized tissue of any kind are as follows:
1. Lack of visible nuclear material in H&E or DAPI-stained sections
2. Total amount of double-stranded DNA less than 0.05 μg/mg dry tissue
3. No individual DNA fragment longer than 200 base pairs

This study focused on the first two criteria by performing H&E staining and DNA quantification, as these tests are more commonly performed and allow for direct comparison to other studies in the field.

Six different treatments of porcine aorta were studied, and the extent of decellularization for each was evaluated using histology and DNA quantification. The treatments were two controls—standard SDS treatment and treatment with dry CO₂—and treatment with supercritical CO₂ and four different additives: pure water, water+Ls-54, pure ethanol, and water+ethanol. The CO₂ treatments were performed at 37° C. and 27.6 MPa (ρ_{CO2}=0.908 g/mL) for about 1 hour with a 0.345 megapascal per minute depressurization rate.

Tissue sections from each treatment were stained with hematoxylin and eosin (H&E) and observed under an optical microscope. Sections from the tunica media of each of the controls can be seen in FIG. 5 at 10× and 40× magnification. In the untreated tissues (images a and d on the Figure), the elastic fibers of the ECM can be readily observed in a parallel orientation with regular spacing between them. Round or elliptical whole smooth muscle cells can be observed attached to the elastic fibers. The middle images (b and e) on the Figure show the tissue after treatment with SDS and PBS washing. Intact cells are not visible, indicating some degree of decellularization. However, dark, irregularly shaped areas of cellular debris are observed, indicating incomplete cell removal. Additionally, significant damage to the ECM fibers is evident based on their widespread breakage and deformation. Tissues treated with dry CO₂ (images c and f) primarily have intact, undisturbed cells compared to the native tissue, though a minority of cells appear to be shriveled or completely removed based on the increased white space in the micrographs. Elastic fibers are disturbed somewhat, as some shrinkage is observed and the spacing between fibers is less uniform, but unlike the SDS treatment, the fibers are not broken entirely.

These findings can be explained by considering the known mechanisms of how detergents and supercritical fluids interact with cells and proteins. The SDS results mirror the literature; it is well-known that most ionic detergents, including SDS, disrupt both the cell and nuclear membranes by replacing molecules in the lipid bilayer via the micelle effect. This effect leads to intracellular contents exiting the confines of the cell and leaving the black splotches of cellular debris found in the micrographs. However, SDS alone will not remove the cellular debris from the matrix; this is usually accomplished by prolonged washing with a saline solution. In this case, a 24-hour PBS wash was performed but likely not for a long enough period of time, as it has been shown that saline rinses often require several days or even weeks to remove all residual cellular material and detergent from a decellularized tissue. It is also well documented that SDS denatures proteins, so the heavy disruption of the elastic fibers is not surprising.

On the other hand, treatment with dry CO₂ was not nearly as disruptive to elastic fibers in the ECM. Though no breakage was observed, there is still a clear loss of uniformity in both fiber size and spacing. This is a reasonable outcome given that tissue dehydration is a known side effect of treatment with dry CO₂. However, CO₂ was ineffective at removing cells from the matrix. This result matches previous observations that CO₂ is ineffective at cell removal without an additive. Though there is currently a clear lack of experimental proof, it has been proposed that the mechanism of CO₂ decellularization is extraction of both whole cells and cellular debris. Because these materials are charged, dissolution in pure CO₂ is minimal because CO₂ is a completely nonpolar molecule. This suggests using a polar, CO₂-soluble additive to aid in decellularization, as described below.

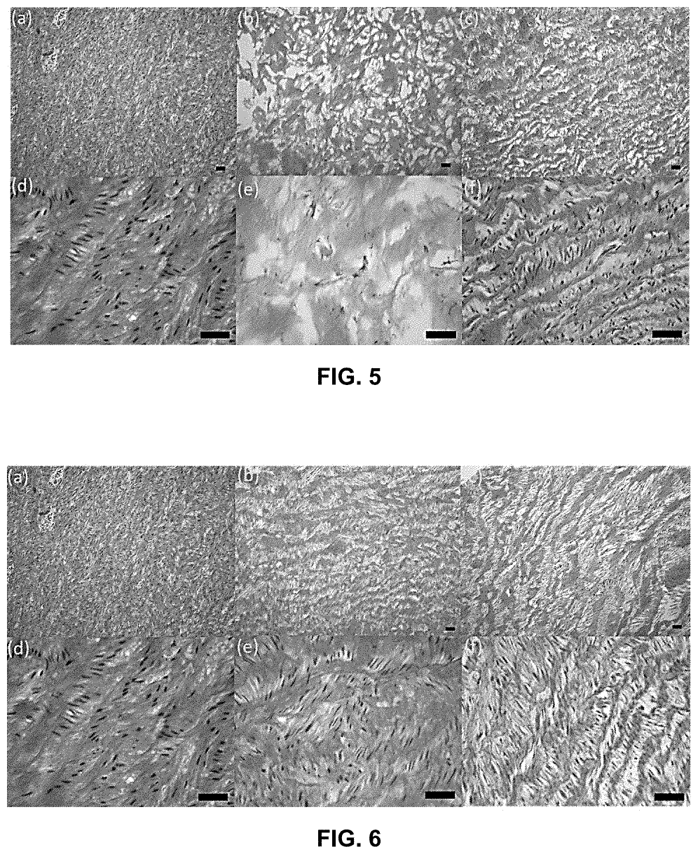

Four different additives were used in an attempt to improve cell removal: (1) water; (2) water plus Ls-54; (3) ethanol; and (4) water plus ethanol. H&E sections from treatments with water/CO₂ (b, e) and water/Ls-54/CO₂ (c, f) are shown in FIG. 6 alongside the untreated tissue (a, d). The results are not noticeably different from those with dry CO₂ (FIG. 5). With regard to decellularization, there appears to be no more effectiveness than with dry CO₂. This is not surprising because although water is polar, it has relatively low solubility in supercritical CO₂, meaning that the humidified CO₂ is still highly nonpolar and is unlikely to be able to extract cells. Using water as an additive does appear to improve the continuity and uniformity of elastic fibers, which makes sense because the tissue is not dehydrated by this particular treatment.

FIG. 6 also shows the effect of adding Ls-54 to the solution to be minimal. Ls-54 is a non-fluorinated surfactant that has been shown in the past to have solubility in supercritical CO₂ and to be an effective CO₂ additive for removing bacterial endotoxin a solid surface. However, it appears to be ineffective in enhancing decellularization. This may be related to its chemistry compared to SDS: Though the chain lengths are similar, Ls-54 contains a hydroxyl group at the end of its chain instead of the highly dissociative sodium ion of SDS. Ls-54 also contains several ethoxyl and propoxyl groups, whereas SDS has a long, nonfunctionalized alkyl chain. These characteristics give SDS distinct hydrophilic and hydrophobic ends that interact with cell membrane lipid bilayers much more effectively than Ls-54.

The ineffectiveness of Ls-54 could also be related to treatment temperature. Past work has shown an inverse proportionality between Ls-54 solubility in CO₂ and temperature, including into the liquid CO₂ phase. The CO₂/water/Ls-54 treatment was also conducted at 10° C. where the CO₂ is liquid (data not shown), but no significant changes in extent of decellularization were observed. While it is generally expected that temperature will affect the performance of decellularization, it is likely that this particular treatment is so far from achieving complete decellularization that these effects cannot be ascertained at the magnification used.

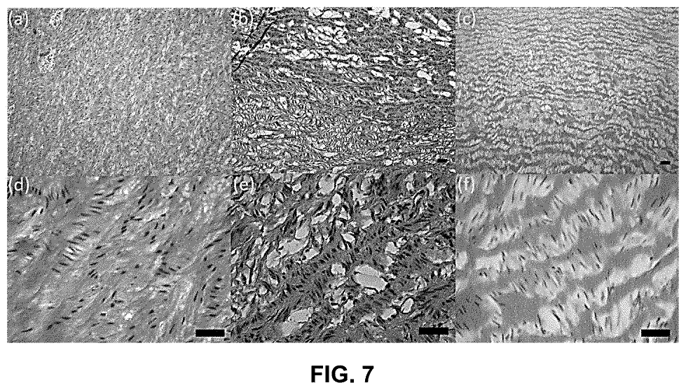

To further increase the polarity of the supercritical CO₂ mixture, two final treatments were performed using ethanol or water and ethanol as additives. H&E sections from treatments with ethanol/CO₂ (b, e) and ethanol/water CO₂ (c, f) are shown in FIG. 7 alongside the untreated tissue (a, d). Treatment with ethanol alone shows considerable shriveling and branching of the elastic fibers, as expected. However, the use of ethanol does not aid considerably in cell removal. Though some of the areas where the ECM is damaged have fewer cells, the intact elastic fibers have numerous intact cells attached to them. The addition of water to the ethanol does not markedly affect the extent of decellularization but does significantly improve the condition of the elastic fibers. This finding is expected based on FIG. 6 and the earlier part of this work; the primary objective of using water as an additive is to prevent dehydration, not to remove cells.

Overall, the three treatments that included water as an additive were notably more effective in maintaining the morphology and alignment of the elastic fibers. This supports the findings presented earlier, which showed that presaturating CO₂ with water before contacting the tissue prevents dehydration of the extracellular matrix during CO₂ treatment. On the contrary, when pure ethanol is the additive, shriveling and fraying of the ECM fibers is observed. These findings were also confirmed visually and by manual handling as treatment clearly increased the rigidity of the matrix when water was not added, while the addition of water maintained the apparent flexibility and pliability of the material. Though interesting, the prevention of tissue dehydration is made impractical by the lack of cellular removal in any of the experiments.

Ultimately, microscopy indicates only very limited cell removal with the four supercritical CO₂ and additive treatments, and not nearly enough to indicate decellularization. To confirm visual microscopy results, quantification of DNA was employed as a measure of decellularization; one of the proposed criteria for establishing decellularization is a double-stranded DNA concentration below 0.05 μg DNA/mg dry tissue.

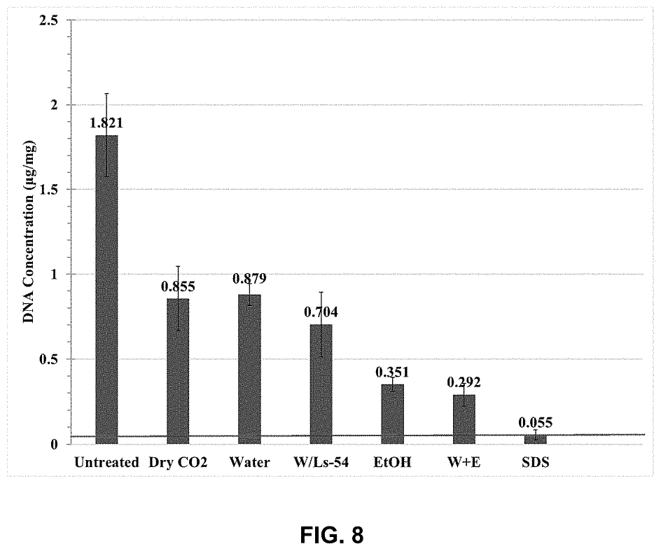

For each treatment in this study, DNA was extracted and its concentration was calculated based on spectrophotometric absorbance readings. Results of DNA quantification are shown in FIG. 8. All treatments show some amount of DNA removal compared to the untreated tissue. However, no treatments aside from the standard SDS method approach the target maximum concentration of 0.05 μg DNA/mg dry tissue.

The DNA results follow the histological findings, where SDS was required in some capacity to rupture cell membranes and attain at least an appreciable amount of cell removal. The four supercritical CO₂ additives do reduce the DNA content compared to the untreated tissue, though none of the treatments approach complete decellularization, as with the H&E findings. The use of ethanol also appears to have the most significant effect on DNA removal compared to supercritical CO₂ with just water or water and Ls-54. However, this is negated by significant dehydration and structural damage as noted above.

The failure of the CO₂/ethanol mixture to decellularize is the most surprising result, given that this finding contrasts with the findings of Sawada, et al. and that the experiments and the apparatuses used in both studies are each very similar. While there may be unknown differences in equipment or specimens that create a significant difference between the studies, and when analyzing the results of an experiment, particularly one where mechanistic steps cannot be viewed in situ, it is imperative to consider the underlying mechanisms to glean information about what is physically occurring during the experiment.

### Hybrid SDS/CO₂ Decellularization

The limited discussion of mechanisms in the literature for supercritical CO₂ decellularization includes two possibilities: 1) supercritical extraction of cells or cellular debris as a primary mechanism, and 2) physical dislodging of cells from the ECM caused by high pressure. Based on our findings, it is not expected that the high pressure alone can remove cells; other work has been published where blood vessels have been decellularized with high hydrostatic pressure—pressures on the order of several hundred megapascals—and cells still require long-term continuous washing to be removed in these applications.

This suggests renewed focus on the extraction mechanism. In the previous section, the ineffectiveness of Ls-54 surfactant in decellularization was discussed, possibly because of its inability to permeate the cell membrane. It was theorized that supercritical CO₂ in general may suffer from this same problem. To test this hypothesis, a two-step hybrid SDS/CO₂ decellularization treatment was investigated. With this treatment, tissues were treated with SDS as described in the Standard Decellularization with SDS section above, but without the subsequent PBS wash. Instead, tissues were then treated (washed) for 1 hour with supercritical CO₂ that was presaturated with ethanol and water at the same thermodynamic conditions used previously.

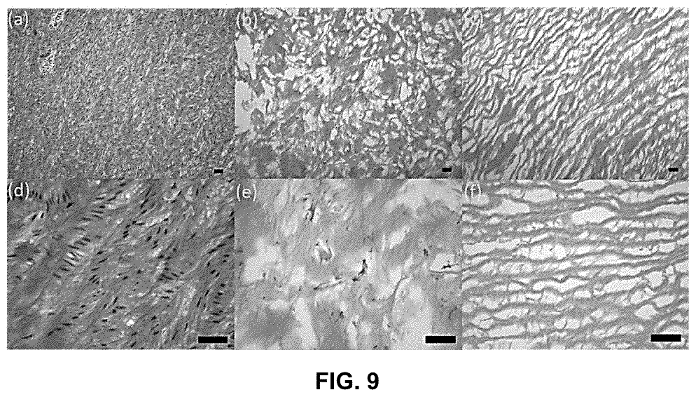

The effect of the hybrid treatment can be seen in FIG. 9, which shows H&E stained sections of untreated (a, d), SDS-treated (b, e), and SDS/CO₂/ethanol/water treated (c, f) tissue samples. Using this hybrid approach shown in images c and f of FIG. 9, there are no visible intact cells or cellular debris. Therefore, it is likely that the tissue is fully decellularized. This suggests polar supercritical CO₂ will extract intracellular debris but will not quickly penetrate the cell membrane to do so unless another agent is used to enhance membrane permeation or rupture. Significantly, the ECM fibers appear to be intact and mostly undisturbed compared to the complete SDS treatment. Maintenance of fiber integrity may be a result of shorter exposure time to SDS; the CO₂/water and ethanol “wash” takes only an hour instead of the day or more required for the standard wash. This also implies that CO₂ treatment removes residual SDS.

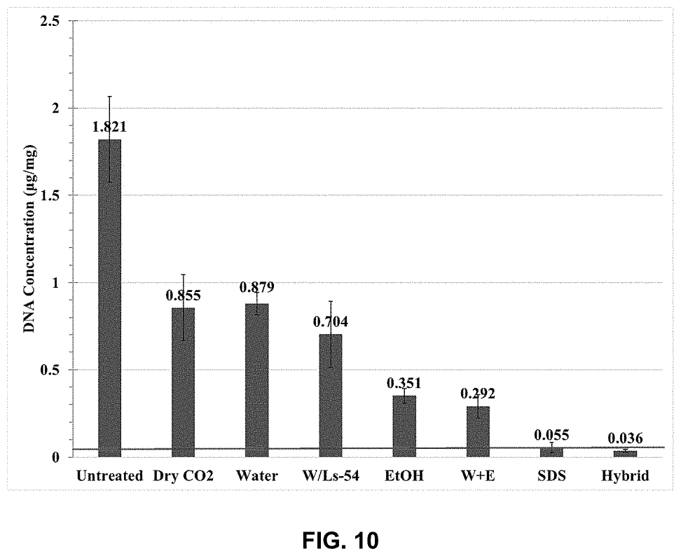

DNA quantification of the hybrid method, along with the results of the other treatments, can be seen in FIG. 10. The Figure shows a level of DNA removal similar to the standard SDS treatment, below the threshold for decellularization with a concentration of 0.036 μg DNA/mg dry tissue. This is a promising result, as the hybrid method is able to achieve the original objective of decellularizing effectively while avoiding dehydration of the tissue.

### Residual SDS Quantitation

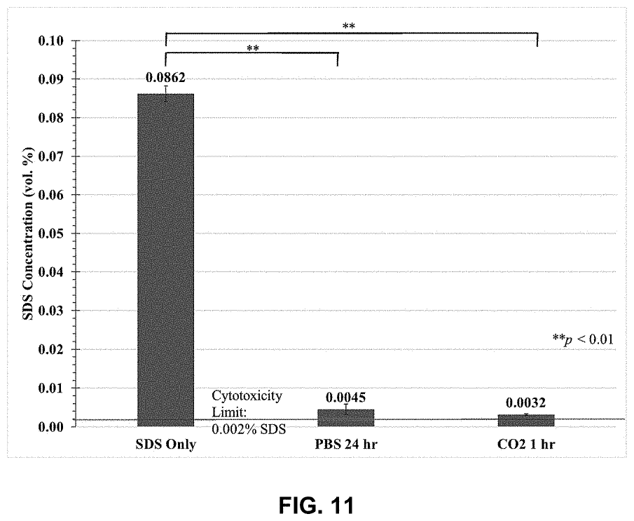

Next, residual SDS from the standard and hybrid treatments was quantified using an SDS Detection and Estimation Kit (G Biosciences, St. Louis, Mo.) as described above. Removal of SDS is a consideration for scaffold viability, as cytotoxicity is observed for many cell types at concentrations greater than about 0.002% SDS. Residual SDS was quantified for the standard SDS treatment and the SDS/supercritical CO₂ hybrid treatment, as shown in FIG. 11. FIG. 11 shows that one hour of supercritical CO₂ treatment removes about as much SDS as 24 hours of washing with PBS, which results in a significant time savings. PBS washes also have diminishing returns, making the wash step last several days in many protocols to reduce SDS below cytotoxic level. Thus, the supercritical CO₂ treatment of the present invention could compare even more favorably over longer time periods. This finding also indicates solubility of SDS in the supercritical CO₂ treatment solution, and this is not surprising since SDS is an organic molecule with similar molecular weight to other molecules extracted by supercritical CO₂, such as caffeine.

### Conclusions

A novel method for decellularizing porcine aorta with supercritical CO₂ and additives while maintaining the native hydration state was presented. First, it was demonstrated that the method reduces or eliminates the extraction of water and other volatiles that has been observed elsewhere. The utility of this method has been verified by experiments on both a model hydrogel and porcine aorta. Even presaturated supercritical CO₂ does extract a small amount of other volatile components from the tissue, potentially lipids and cholesterol, though further analysis is required to verify this. From these observations, it is concluded that presaturation of CO₂ could be used to prevent undesired dehydration of biomaterials for tissue engineering.

After verifying the efficacy of presaturation, the method was used to decellularize porcine aorta. As anticipated, nonpolar CO₂ solutions were proven ineffective for decellularizing porcine aorta by both histology and DNA quantification, though presaturating CO₂ with water did better maintain the hydration state of the matrix, even in the presence of other additives. More surprisingly, the addition of ethanol to increase the CO₂ polarity did not substantially intensify the extent of decellularization, suggesting that CO₂ alone is unable to quickly penetrate the cell membrane and the previously proposed mechanism of whole-cell extraction is unlikely to be valid.

The inability of supercritical CO₂ alone to disrupt cell membranes was further tested by the development of a hybrid decellularization protocol that utilized a short SDS pretreatment step before washing with CO₂ and water and ethanol additives. This treatment shows that CO₂ can extract intracellular material if the cell membrane is lysed beforehand. Complete decellularization was achieved using this method while maintaining the hydration state of the native tissue. Further study is required to determine the capabilities and limitations of this method, particularly regarding maintenance of the mechanical properties of the matrix.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only and is not intended to limit the invention so further described in the appended claims.

What is claimed is:

1. A method for decellularizing tissue, the method comprising:
placing the tissue in a pretreatment chamber;
pretreating the tissue with a surfactant under agitation in the pretreatment chamber; forming a decellularization solution in a presaturation chamber in an environmental chamber that is separate from the pretreatment chamber, wherein the decellularization solution comprises carbon dioxide and one or more polar solvents at a temperature greater than 31.1° C., wherein the carbon dioxide is maintained at a pressure greater than 7.38 megapascals to form supercritical carbon dioxide;
placing the tissue in a treatment chamber located in the environmental chamber; and treating the tissue with the decellularization solution from the presaturation chamber.

2. The method of claim 1, wherein a pump compresses the carbon dioxide before the carbon dioxide is delivered to the tissue.

3. The method of claim 1, wherein the carbon dioxide and the one or more polar solvents are mixed for a time period ranging from about 1 minute to about 30 minutes prior to exposing the tissue to the decellularization solution.

4. The method of claim 1, wherein the one or more polar solvents comprises ethanol, methanol, isopropanol, water, acetic acid, or a combination thereof.

5. The method of claim 1, wherein the decellularization solution is mixed in the presaturation chamber for a time period ranging from about 1 minute to about 30 minutes.

6. The method of claim 1, wherein the decellularization solution is delivered to the treatment chamber at a flow rate ranging from about 0.1 millimeters per minute to about 5 milliliters per minute.

7. The method of claim 1, wherein the tissue is exposed to the decellularization solution for a time period ranging from about 1 minute to about 2 hours.

8. The method of claim 1, wherein the surfactant comprises sodium dodecyl sulfate.

9. The method of claim 9, wherein the method facilitates removal of cells from the tissue so that tissue treated with decellularization solution contains less than 0.05 micrograms of DNA per milligram of dry tissue after the tissue is exposed to the decellularization solution.

10. The method of claim 1, wherein the system is depressurized at a rate ranging from about 0.1 megapascals per minute to about 0.6 megapascals per minute.

11. The method of claim 1, wherein the tissue treated with the decellularization solution contains from about 0.001 volume % to about 0.004 volume % of surfactant after the tissue is exposed to the decellularization solution.

12. The method of claim 1, wherein the tissue is pretreated with the surfactant in the pretreatment chamber for a time period of up to 48 hours.

13. The method of claim 1, wherein the carbon dioxide is continually mixed with the one or more polar solvents.
